(19) United States
(12) Patent Application Publication
(10) Pub. No.: US 2004/0013723 A1
(43) Pub. Date: Jan. 22, 2004

(54) Oral care capsules

(76) Inventors: Rita M. Parikh, Paramus, NJ (US); Lori Dee Kumar, Skillman, NJ (US)

Correspondence Address: Darryl C. Little, Attorney for Applicant, Warner Lambert Company, 201 Tabor Road, Morris Plains, NJ, 07950, US

(21) Appl. No.: 10/406,851

(22) Filed: Apr. 4, 2003

Related U.S. Application Data
(60) Provisional application No. 60/396,401, filed on Jul. 16, 2002.

(51) Int. Cl. A61K 9/48; A61K 9/64; A61K 9/16; A61K 9/50

(52) U.S. Cl. 424/456; 424/490

(57) ABSTRACT

This invention is concerned with improvements in and relating to soft gelatin capsules or microcapsules. More particularly, it is concerned with oral care capsules or microcapsules providing improved biological or therapeutic activity.

This application claims the benefit of U.S. Provisional Patent Application No. 60,396,401, filed on Jul. 16, 2002, the entirety of which is hereby incorporated by reference as if fully set forth herein.

### FIELD OF THE INVENTION

This invention is concerned with improvements in and relating to soft gelatin capsules or microcapsules. More particularly, it is concerned with oral care capsules or microcapsules providing improved biological or therapeutic activity.

### BACKGROUND OF THE INVENTION

Pharmaceutical compositions in dosage unit form encapsulated in soft gelatin capsules are well known and generally consist of a fill material comprising one or more active agents dissolved or suspended in an appropriate liquid or paste vehicle, encapsulated in a soft gelatin shell, typically comprising gelatin together with a plasticizer. Manufacture of soft gelatin capsules requires the fill material to be a pumpable liquid or paste. The carrier liquid can be a single or a multi-component system that must be compatible with the soft gelatin capsule.

Liquids used in soft gelatin capsules fall into two general categories, hydrophilic and lipophilic. There are few hydrophilic liquids suitable for use as carrier liquids in this application. The most versatile being polyalcohols and short chain glycols such as polyethylene glycols (particularly in the molecular weight range of 200-800). These materials offer good dispersion in gastric medium, excellent solubilizing capabilities for pharmaceutical active ingredients and good compatibility in the soft gelatin capsule format. However, there are disadvantages to using these materials. One major disadvantage is that of instability; while in the presence of atmospheric oxygen these compounds react to form aldehydes. The residual aldehyde content can react with the gelatin shell causing the protein polymers to inter- and intra-crosslink. The net result is a crosslinked gelatin shell having poor dissolution properties and becoming brittle. These disadvantages can be overcome by using lipophilic liquids.

Regarding lipophilic fill components, there are many acceptable examples. Usually these are oils and are not water soluble. Typical examples include mineral oils (petroleum or petroleum-derived), vegetable oils (chiefly from seeds and nuts), animal oils (usually occurring as fats; the liquid types include fish oils), edible oils (chiefly vegetable oils as well as some special fish oils) and triglycerides (preferably short chain triglycerides). These components, however, can pose a problem where the active is also an oil or an oil soluble component. A prime example involves the use of essential oils as antimicrobial actives. Without being limited by theory, it is believed that lipophilic fill materials, especially fixed or higher molecular weight oils (molecular weight range of greater than about 250) tend to bind or partition the oily actives, in many instances, to the extent that the therapeutic or biologic activity of the essential oil active is substantially decreased or inhibited.

Therefore, there is a need for fill ingredient components free of properties which might inhibit or reduce the therapeutic or biologic activity of essential oil actives.

The present inventors have discovered that by incorporating therapeutically or biologically active (e.g., antimicrobially active) essential oils in capsules or microcapsules with substantially reduced amounts of fixed oils, the therapeutically or biologically active essential oil remains substantially free to exert its therapeutic or biologic activity.

It is therefore an aspect of the present invention to provide improved oral dosage forms.

It is another aspect of the present invention to provide improved oral dosage forms for use in the oral cavity.

It is another aspect of the present invention to provide oral dosage forms which improve the availability of therapeutically or biologically active essential oils.

It is another aspect of the present invention to reduce the binding or partitioning of therapeutically or biologically active essential oil in fixed oils by incorporating at least one additional essential oil.

It is another aspect of the present invention to provide capsules or microcapsules that provide improved breath control and antimicrobial activity.

Another aspect of the present invention is to provide improved methods of providing breath control and reduction in oral bacteria.

These and other aspects of the present invention will become more apparent from the detailed description that follows.

### SUMMARY OF THE INVENTION

The present invention in one of its aspects relates to oral capsule or microcapsules, comprising:

a. a shell; and

b. a core, comprising:

i.) at least one therapeutically or biologically active essential oil; and

ii.) less than about 20%, by weight of the total capsule or microcapsule, of a fixed oil.

In another embodiment of the present invention relates to an oral capsule or microcapsule, comprising:

a. a shell; and

b. a core, comprising:

i.) an antimicrobially effective amount of at least one antimicrobially active essential oil;

ii.) less than about 20%, by weight of the total capsule or microcapsule, of a fixed oil; and

iii.) greater than about 10%, by weight of the total capsule or microcapsule, of at least one additional essential oil.

Methods of using such compositions as a carrier for systemic or oral care actives are also disclosed.

All percentages and ratios used herein are by weight of the total capsule or microcapsule unless otherwise specified. Additionally, all measurements are made at 25° C. unless otherwise specified.

The compositions of the present invention can comprise, consist essentially of, or consist of, the essential as well as optional ingredients and components described herein. As used herein, “consisting essentially of” means that the composition or component may include additional ingredients, but only if the additional ingredients do not materially alter the basic and novel characteristics of the claimed compositions or methods.

The term “rapidly (or fast) dissolving” as used herein means that the microcapsule dissolves in less than about 60 seconds, preferably less than about 30 seconds, more preferably less than about 15 seconds, after placing the microcapsule in the oral cavity.

### DETAILED DESCRIPTION OF THE INVENTION

The essential as well as optional components of the capsules of the present invention are described in the following paragraphs.

Capsule Shell Material

The capsule or microcapsule shells of the present invention are manufactured using conventional capsule manufacturing technology. The shell material of the microcapsules of the present invention can be any materials which are suitable for ingestion as well as retention in the oral cavity. Materials which are suitable include gelatin, polyvinyl alcohols, waxes, gums, sucrose esters, pullulan and sugar candy type materials used in cough drops and mints, for example. For a general description of gelatin and gelatin-based capsules, see Remington's Pharmaceutical Sciences. 16^{th} ed., Mack Publishing Company, Pa. (1980), page 1245 and pages 1576-1582. Additional materials and capsule manufacturing technologies can be found in U.S. Pat. No. 2,800,458; 3,159,585; 3,533,958; 3,697,437; 3,888,689; 3,996,156; 3,965,033; 4,010,038; and 4,016,098, each of which are herein incorporated by reference in their entirety.

The shell or wall content of the microcapsules comprises from about 1% to about 25%, preferably from about 5% to about 16%, most preferably from about 5% to about 10%, by weight of the capsule or microcapsule. The shell material is used to form any of a wide variety of shapes such as spheres, oblong shapes, disks, puffed squares and cylinders. The shell thickness is preferably in the range of about 30 um to about 2 mm, preferably from about 70 um to about 110 um. If the microcapsules are spherical, the particle diameter is generally in the range of from about 2 mm to about 9 mm, preferably from about 3 mm to about 7 mm. Additional disclosure regarding the shell component of the present invention can by found in U.S. Pat. Nos. 5,332,584 and 5,126,061, both of which are herein incorporated by reference.

Core Materials

The compositions of the present invention further comprise from about 75% to about 99%, preferably from about 84% to about 95%, and more preferably from about 90% to about 95% by weight of the capsule or microcapsule of core materials. Such core materials include the following:

A Biologially or Therapeutially Active Essential Oil

Particularly preferred for use herein are essential oils capable of providing biologic or therapeutic activity, especially antimicrobial activity, in the oral cavity. Such antimicrobially effective oils include, but are not limited to, Cedarwood oil (China) BP, Camphor oil (White), Camphor powder synthetic technical, Cardamom oil, Cinnamon bark oil, Cinnamon leaf oil, Citronella oil, Clove bud oil, Clove leaf, Ginger oil, Ginger oleoresin (India), I-Carvone, Citral, Geraniol, Geranyl Acetate, Geranyl Nitrile, Grapefruit oils, Hydroxycitronellal, Menthol, Eucalyptol, Thymol, Methyl Salicylate, Tea tree oil, Terpineol, Linalool, Nerol, and mixtures thereof. Preferred biologically or therapeutically active essential oils include Menthol, Eucalyptol, Thymol, Methyl Salicylate and mixtures thereof.

In the capsules or microcapsules of this invention, the essential oils are used in amounts effective to provide biologic or therapeutic activity in the oral cavity. Generally, the total amount of essential oils present in the capsules or microcapsules can be from about 1% to about 50% w/w, optionally from about 5.0% to about 45%, or, optionally, from about 10% to about 30%.

Thymol is preferably employed in the microcapsules of this invention in amounts of from about 0.001% to about 15% w/w, and most preferably from about 0.01% to about 8% w/w. Eucalyptol is preferably employed in amounts of from about 0.001% to about 15% w/w, and most preferably from about 0.01% to about 10% w/w. Menthol is preferably employed in amounts of from about 0.1% to about 25% w/w, most preferably from about 1% to about 15% w/w. Methyl salicylate is preferably employed in amounts of from about 0.001% to about 15% w/w, and most preferably from about 0.01% to about 10% w/w.

Fixed Oil

Particularly preferred components for use herein are fixed oils. As used herein, “fixed oils” are nonvolatile, fatty oils characteristic of vegetable oils as opposed to essential oils of plants. As used herein, fixed oils also includes triglycerides.

Examples of suitable fixed oils and triglycerides can be found in U.S. Pat. No. 4,935,243, herein incorporated by reference in its entirety. Preferred fixed oils include, but are not limited to, corn, olive, rapeseed, sesame, peanut, sunflower, safflower, vegetable, or mineral oils. Preferred triglycerides include, but are not limited to, capric/caprylic triglycerides (e.g., Neobee M5 [Stepan Chemical—Northfield, Ill.] and Captex 300 [Karlshams Lipid Specialties—Columbus Ohio]; distilled succinylated monoglycerides of fatty acids such as the Myverol product series (Eastman Chemicals Co.); stearate esters (Lipo) and polyethylene glycols such as PEG 400. These materials are described in further detail in U.S. Pat. Nos. 6,117,835; 6,096,338; 6,083,430; and 6,045,835, each of which are herein incorporated by reference in their entirety. Preferably the fixed oils are present invention at concentrations of less than about 20%, more preferably less than about 15%, and most preferably less than about 10% of the total capsule or microcapsule weight. Mixtures of the above fixed oils (including triglycerides) can also by used.

Optional Ingredients

Additional Essential Oils

Optionally and preferred for use in the core of the capsules or microcapsules of the present invention are additional essential oils other than the biologically or therapeutically active essential oils. Essential oils are generally described as complex volatile liquids derived from flowers, stems, and leaves and often the entire plant. As used herein, the term “essential oils” also includes artificial or synthetic oils having similar or substantially similar properties. The essential oils of the present invention preferably have the following molecular characteristics: a.) number average molecular weight of less than about 250, preferably less than about 200, most preferably less than about 175; b.) a hydration energy of less than 4 kCal/mol.; c.) a molecular surface area of less than 700 Å², preferably less than 550 Å²; and d.) a molecular volume of less than 1000 Å³, preferably less than 860 Å³.

Suitable additional essential oils include, but are not limited to, Almond bitter, Amyris, Anise, Anise (Star), Anethole 20/21 natural, Aniseed oil china star, Aniseed oil globe brand, Balsam (Peru), Basil oil, Bay (Myrcia), Bergamot oil, Birch Bark oil, Bois de rose oil, Black pepper oil, Black pepper oleoresin 40/20, Bois de Rose (Brazil) FOB, Borneol Flakes (China), Cananga oil (Java), Caraway, Cassia oil (China), Coriander (Russia), Coumarin 69° C. (China), Cyclamen Aldehyde, Diphenyl oxide, Ethyl vanilin, Eucalyptus citriodora, Fennel oil, Geranium oil, White grapefruit oil, Guaiacwood oil, Gurjun balsam, Heliotropin, Isobomyl acetate, Isolongifolene, Jasmine oil, Juniper berry oil, Labdanum oil, L-methyl acetate, Lavandin oil, Lavender oil, Lemon oil, Lemongrass oil, Lime oil distilled, Litsea Cubeba oil, Longifolene, Methyl cedryl ketone, Methyl chavicol, Mint (Japanese) oil, Musk ambrette, Musk ketone, Musk xylol, Neroli oil, Nutmeg oil, Ocotea (cynbarum) oil, Orange (bitter), Orange (sweet), Origanum oil, Orris root oil, Palmarosa, oil, Patchouli oil, Peppermint oil, Phenyl ethyl alcohol, Pimento berry oil, Pimento leaf oil, Rosalin, Sandalwood oil, Sandenol, Sage oil, Clary sage, Sassafras oil, Spearmint oil, Spike lavender, Tagetes, Vanilin, Vetyver oil (Java), Wintergreen, Allocimene, Arbanex TM, Arbanol Registered TM, Bergamot oils, Camphene, Alpha-Campholenic aldehyde, Cineoles, Citronellol Terpenes, Alpha-Citronellol, Citronellyl Acetate, Citronellyl Nitrile, Para-Cymene, Dihydroanethole, Dihydrocarveol, d-Dihydrocarvone, Dihydrolinalool, Dihydromyrcene, Dihydromyrcenol, Dihydromyrcenyl Acetate, Dihydroterpineol, Dimethyloctanal, Dimethyloctanol, Dimethyloctanyl Acetate, Estragole, Ethyl-2 Methylbutyrate, Fenchol, Fernlol TM, Florilys TM, Gildmint TM Mint oils, Glidox TM, trans-2-Hexenal, trans-2-Hexenol, cis-3-Hexenyl Isovalerate, cis-3-Hexanyl-2-methylbutyrate, Hexyl Isovalerate, Hexyl-2-methytbutyrate, Ionone, Isobomyl Methylether, Linalool Oxide, Linalyl Acetate, Menthane Hydroperoxide, I-Methyl Acetate, Methyl Hexyl Ether, Methyl-2-methylbutyrate, 2-Methylbutyl Isovalerate, Myrcene, Neryl Acetate, 3-Octanol, 3-Octyl Acetate, Phenyl Ethyl-2-methylbutyrate, Petitgrain oil, cis-Pinane, Pinane Hydroperoxide, Pinanol, Pine Ester, Pine Needle oils, Pine oil, alpha-Pinene, beta-Pinene, alpha-Pinene Oxide, Plinol, Plinyl Acetate, Pseudo Ionone, Rhodinol, Rhodinyl Acetate, Spice oils, alpha-Terpinene, gamma-Terpinene, Terpinene-4-OL, Terpinolene, Terpinyl Acetate, Tetrahydrolinalool, Tetrahydrolinalyl Acetate, Tetrahydromyrcenol, Tetralol Registered TM, Tomato oils, Vitalizair, Zestoral TM or mixtures thereof.

When used as an additional component (i.e., in combination with a biologically or therapeutically active essential oil), the essential oil component is preferably present at concentrations of greater than about 10%, more preferably from about 15% to about 50%, and most preferably from about 20% to about 50%.

Humectants

Also optionally useful in the capsules or microcapsules of the present invention, among other things, as a plasticizer are humectants. Humectants serve to retain water on/in the surfaces of the oral cavity. Examples of suitable humectants include polyhydric alcohols selected from the group consisting of ethylene glycol, propylene glycol, dipropylene glycol, butylene glycol, hexylene glycol, polyethylene glycols, glycerin sorbitol, panthenols, urea, alkoxylated glucose derivatives, such as Glucam (RTM) E-20, hexanetriol, glucose ethers, sodium hyaluronate, soluble chitosan and mixtures thereof. Glycerin and/or sorbitol are presently preferred.

The sorbitol used in the invention is sold by the Company Roquette under the trade name Neosorb P 60 W or Neosorb p-60. The glycerin used in this invention is preferably “glycerin, USP, 99.5%”, most preferably that which is sold by Dow Chemical, Inc., Emery Industries, Inc. (under the name “Superol 99.5%”), and Procter & Gamble.

Humectants are preferably present in the capsules or microcapsules of the present invention at concentrations of from about 0.01% to about 12%, preferably from about 0.5% to about 8%, more preferably from about 1% to about 6%.

Additional Optional Components

The capsules or microcapsules of this invention may also contain any number of additional materials in either the shell and/or core to provide additional breath freshening efficacy and/or sensory perceptions. Such agents may include quaternary ammonium salts such as pyridinium salts (e.g., cetyl pyridinium chloride), domiphen bromide, other cationic materials such as chlorhexidine salts, zinc salts and copper salts (particularly copper gluconate). Suitable and preferred copper and zinc salts can be found in U.S. Pat. Nos. 5,628,986 and 6,121,315, respectively, both of which references are herein incorporated by reference in its entirety. Other agents such as phenolics, chlorhexidine, triclosan, peroxides, povidone-iodine, chlorine dioxide, neem, wild indigo, barberry, green tea, calendula, fennel, golden seal, chaparrel, chamomile, propolis, thyme, calendula as well as additional noncationic water insoluble agents are also useful herein. Such materials are disclosed in U.S. Pat. No. 5,043,154, Aug. 27, 1991, incorporated herein by reference in its entirety. Mixtures of the above mentioned breath control/antimicrobial agents may also be used. These breath control/antimicrobial agents are used in an amount of from about 0.001% to about 2%, preferably from about 0.005% to about 1% of the total composition.

Antimalodorants useful in the present invention at levels necessary to produce the satisfactory masking of mouth malodor and include, but are not limited to, zinc salts, copper salts, chlorophyllins, apha ionones, geraniol, parsley seed and mixtures thereof.

Fluoride providing compounds may be present in the capsules or microcapsules of this invention. These compounds may be slightly water soluble or may be fully water soluble and are characterized by their ability to release fluoride ions or fluoride containing ions in water. Typical fluoride providing compounds are inorganic fluoride salts such as amine fluorides, alkali+fluoride, ammonium fluoride, cuprous fluoride, zinc fluoride, stannic fluoride, stannous fluoride, barium fluoride, sodium fluorozirconate, sodium monofluorophosphate, aluminum mono- and difluorophosphate, fluorinated sodium calcium pyrophosphate, acidulated monofluorophosphate and mixtures thereof.

Alkali metal, tin fluoride and monofluorophosphates such as sodium and stannous fluoride, sodium monofluorophosphate and mixtures thereof are preferred.

In the capsules or microcapsules of the present invention, the fluoride providing compound is generally present in an amount sufficient to release up to about 0.15%, preferably about 0.0005% to about 0.1% and most preferably from about 0.001% to about 0.05% fluoride by weight of the preparation.

Additionally, a variety of sweetening agents may also be included into either the core and/or shell of the capsules or microcapsules described herein. Suitable sweeteners may be selected from the following non-limiting list: sugars such as sucrose, glucose (corn syrup), dextrose, invert sugar, fructose, and mixtures thereof, chlorodeoxysucrose derivatives (such as described in U.S. Pat. Nos. 4,343,934, 4,435,440 and 4,389,394, each of which is herein incorporated by reference in its entirety); saccharin and its various salts such as the sodium or calcium salt; cyclamic acid and its various salts such as the sodium salt; the dipeptide sweeteners such as aspartame; dihydrochalcone compounds, glycyrrhizin; Stevia Rebaudiana (Stevioside); glycyrrhizin, dipotassium glycyrrhizin, phenylalanine 1-methyl ester (Aspartame); chloro derivatives of sucrose; dihydroflavinol; hydroxyguaiacol esters; L-amino dicarboxylic acid gem-diamines; L-aminodicarboxylic acid aminoalkenoic acid ester amides; and sugar alcohols such as sorbitol, sorbitol syrup, mannitol, xylitol, and the like. Also contemplated as an additional sweetener is the nonfermentable sugar substitute (hydrogenated starch hydrolysate) which is described in U.S. Pat. No. Re. 26,959. Also contemplated is the synthetic sweetener 3,6-dihydro-6-methyl1-1-1,2,3 -oxathiazin-4-one-2,2-dioxide, particularly the potassium (acesulfame-K), L-alpha-Aspartyl-N-(2,2,4,4-tatramethyl-3-thietanyl)-D-alaninamide hydrate (Alitame, a commercially available product of Pfizer, New York, N.Y.); and thaumatin (Talin).

These agents are used in an amount of from about 0.1% to about 10%, preferably from about 0.35% to about 3% of the total capsule weight. A more detailed discussion of additional as well as preferred sweetening and taste/flavor modifying materials can be found in U.S. Pat. Nos. 6,121,315 and 5,284,659, both of which are herein incorporated by reference in their entirety. Mixtures of any of the additionally disclosed sweeteners can also be used.

Particularly preferred for use in the present invention, in combination with the chlorodeoxy sucrose derivative, is acesulfame. Acesulfame is the synthetic sweetener 3,6-dihyro-6-methyl1-1-1,2,3-oxathiazin-4-one-2,2-dioxide and is, generally, incorporated into the capsules or microcapsules of the present invention as acesulfame K (Sunnett Brand Sweetener available from Hoechst Celanes, Portsmouth, Va.). Preferably the chlorodeoxysucrose derivative and acesulfame are combined at a ratio of from about 1:1 to about 9:1, more preferably from about 2:1 to about 7:3.

Vitamins such as vitamin A (retinol and carotene derivatives); vitamin B (thiamine, riboflavin, niacin, panthothenic acid, biotin, cyanocobalamin, pyridoxine, folic acid, inositol); vitamin C (ascorbic acid); vitamin D (ergocalciferol, cholecalciferol, ergosterol); vitamin E (tocopherol); vitamin K (phytonadione, menadione, phthiocol) as well as other and more specific antioxidants can also be incorporated into the capsules or microcapsules of the present invention. Suitable as well as preferred vitamins and antioxidants can be found in U.S. Pat. No. 6,238,678, herein incorporated by reference in its entirety.

The capsules or microcapsules of the present invention may also contain one or more sensory or sensate actives to act as warming or cooling signals.

When used in the present invention, sensates or sensory actives can be present at a level of from about 0.01% to about 10%, typically from about 0.1% to about 5%, and preferably from about 0.2% to about 1%. The level is selected to provide the desired level of consumer perceived sensation and can be modified as desired. Suitable sensate technologies include mannitol, inositol, physcool®, menthol, eucalyptus, 3-1-menthoxy propane-1,2-diol, N-substituted-p-menthane-3-carboxamides and acyclic carboxamides.

3-1-menthoxy propane 1,2-diol is fully described in detail in U.S. Pat. No. 4,459,425, issued Jul. 10, 1984 to Amano et. al, incorporated herein by reference in its entirety. This volatile aromatic is commercially available, being sold by Takasago Perfumery Co., Ltd., Tokyo, Japan.

The N-substituted-p-menthane-3-carboxamides are fully described in U.S. Pat. No. 4,136,163 to Watson et al., issued Jan. 23, 1979 incorporated herein by reference in its entirety. The most preferred volatile aromatic of this class is N-ethyl-p-menthane-3-carboxamide which is commercially available as WS-3 from Wilkinson Sword Limited.

Useful acyclic carboxamides are fully described in U.S. Pat. No. 4,230,688 to Rowsell et al., issued Oct. 28, 1980 incorporated herein by reference in its entirety. The most preferred volatile aromatic of this class is N,2,3-trimethyl-2-isopropylbutanamide which is commercially available as WS-23 from Wilkinson Sword Limited.

Suitable warming type sensory or sensate actives include anhydrous PEG, vanillyl alcohol n-butyl ether (TK-1000 supplied by Takasago Perfumery Co., Ltd., Tokyo, Japan), vanillyl alcohol n-propyl ether, vanillyl alcohol isopropyl ether, vanillyl alcohol isobutyl ether, vanillyl alcohol n-amino ether, vanillyl alcohol isoamyl ether, vanillyl alcohol n-hexyl ether, vanillyl alcohol methyl ether, vanillyl alcohol ethyl ether, gingerol, shogaol, paradol, zingerone, capsaicin, dihydrocapsaicin, nordihydrocapsaicin, homocapsaicin, homodihydrocapsaicin, ethanol, iso-propyl alcohol, iso-amylalcohol, benzyl alcohol and mixtures thereof.

Mixtures of any of the above sensory actives or sensates can also be used.

The capsules or microcapsules of the present invention may also contain sialogogues or agents that stimulate the secretion of saliva. Such agents include, but are not limited to, ascorbic acid, fumaric acid, citric acid, tartaric acid, malic acid, gluconic acid, pilocarpine, mayweed (akkal-kadha), echinacea, coleus, gentian, prickly ash, licorice, ginger, yerba santa, cardomom, monosodium glutamate and mixtures thereof.

Mucoadhesive or bioadhesives are also useful herein. Such agents include, but are not limited to, polyethylene oxide homopolymer, Carbopol®, Plasdone®, CMC, HEC, Klucel®, hydroxypropyl methylcellulose, Gantrez®, polyacrylates and mixtures thereof. These and other suitable muco- or bioadhesives along with preferred ones are detailed in U.S. Pat. Nos. 4,900,522; 5,284,659; 5,458,879; 5,989,535; 6,177,096; 6,200,604; 6,207,180; 6,210,705; 6,213,126; each of which is herein incorporated by reference in its entirety.

The compositions of this invention may also contain coloring agents or colorants. The coloring agents are used in amounts effective to produce the desired color. The coloring agents (colorants) useful in the present invention include natural foods colors and dyes suitable for food, drug and cosmetic applications. These colorants are known as F.D. & C. dyes and lakes. The materials acceptable for the foregoing spectrum of use are preferably water-soluble, and include indigiod dye, known as F.D. & C. Blue No. 2, which is the disodium salt of 5,5-indigotindisulfonic acid. Similarly, the dye known as Green No. 1 comprises a triphenylmethane dye and is the monosodium salt of 4-[4-N-ethyl-p-sulfobenzylamino)diphenylmethylene]-[1-N-ethyl-N-p-sulfoniumbenzyl)-D.sup.2,5-cyclohexadienimine]. Additional examples include the yellow dye, known as D &C Yellow No. 10, and the dye known as F.D. & C. Green No. 3 which comprises a triphenylmethane dye. A full recitation of all F.D. & C. and D. & C. dyes and their corresponding chemical structures may be found in the Kirk-Othmer Encyclopedia of Chemical Technology, Volume 5, pages 857-884, which text is accordingly incorporated herein by reference.

Water or hydroalcoholic mixtures can also present in the capsules or microcapsules of the present invention. Water comprises from about 0.1% to about 15%, preferably from about 1% to about 10%, more preferably from about 1% to about 7% of the capsules or microcapsules described herein. These amounts of water include the free water which is added, plus that amount which is introduced with other materials such as with sorbitol. The water, used in the present invention should preferably be deionized, distilled, flee of organic impurities and bacteria and substantially free of metal ions.

Any of the ingredients mentioned herein for use in the present invention may be incorporated into the shell and/or core of the disclosed capsules or microcapsules.

Method of Manufacture

The capsules or microcapsules of the present invention can be made using a variety of conventional techniques. One method is described after the following examples.

Industrial Applicability:

The capsules or microcapsules of the present invention are used by placing the capsules or microcapsules into the mouth and retaining them therein for a period sufficient to provide the desired effect.

The following examples further describe and demonstrate preferred embodiments within the scope of the present invention. The examples are given solely for the purposes of illustration and are not to be construed as illustrative of limitations of this invention. Many variations thereof are possible without departing from the invention's spirit and scope.

### EXAMPLES

The following compositions are representative of the present invention.

| | Ex. 1 | Ex. 2 | Ex. 3 | Ex. 4 |
|---|---|---|---|---|
| Component | % w/w | % w/w | % w/w | % w/w |
| Gelatin | 12.570 | 12.320 | 15.070 | 5.250 |
| Sorbitol | — | — | 2.060 | 2.050 |
| Acesulfame | — | — | 0.1690 | 0.1920 |
| Potassium | | | | |
| Sucralose | 0.700 | 0.4490 | 0.641 | 0.700 |
| Glycerin | — | — | 2.04 | 2.04 |
| Color | 0.001-.01 | 0.001-.01 | 0.001-.01 | 0.001-.01 |
| Water | — | — | 0.600 | 0.575 |
| Thymol | 5.24 | 5.24 | 2.250 | 1.642 |
| Methyl | 6.99 | 6.99 | 3.068 | 1.400 |
| Salicylate | | | | |
| Eucalyptol | 7.80 | 7.80 | 4.172 | 1.540 |
| Menthol | 10.49 | 10.49 | 16.159 | 21.522 |
| Flavor Oil | 39.31 | 39.611 | 41.671 | 37.989 |
| Neobee M-5 | 15.80 | 17.00 | 12.00 | 18.00 |

The above compositions are prepared by mixing the components of the core in one container and the components of the shell(s) in another container. The shell(s) materials are heated to provide a fluid medium. The core and shell(s) materials are then pumped separately to a two or three fluid nozzle submerged in an organic carrier medium. The capsules formed are allowed to cool and stiffen. They are then denatured and separated for further handling.

In the above compositions any of a wide variety of other shell materials, breath control agents, sweeteners as well as other components may be used in place of or in combination with the components listed above.

What is claimed is:

1. A capsule or microcapsule, comprising:
a. a shell; and
b. a core, comprising:
i.) at least one therapeutically or biologically active essential oil; and
ii.) less than about 20%, by weight of the total capsule or microcapsule, of a fixed oil.

2. A capsule or microcapsule according to claim 1, wherein the shell material is selected from the group consisting of polyvinyl alcohol, gelatin, pullulan, waxes, gums and sugar candies.

3. A capsule or microcapsule according to claim 2, wherein the shell material is gelatin.

4. A capsule or microcapsule according to claim 1, wherein the capsule or microcapsule is in the form of a sphere or an oblong.

5. A capsule or microcapsule according to claim 4, wherein the capsule or microcapsule is in the form of spheres.

6. A capsule or microcapsule according to claim 5, wherein the capsule or microcapsule is from about 2 mm to about 9 mm in diameter and the shell wall thickness is from about 30 um to about 2 mm.

7. A capsule or microcapsule according to claim 1, further comprising a plasticizer.

8. A capsule or microcapsule according to claim 7, wherein the plasticizer is a humectant selected from the group consisting of ethylene glycol, propylene glycol, dipropylene glycol, butylene glycol, hexylene glycol, polyethylene glycols, glycerin sorbitol, panthenols, urea, alkoxylated glucose derivatives, hexanetriol, glucose ethers, sodium hyaluronate, soluble chitosan and mixtures thereof.

10. A capsule or microcapsule according to claim 1, wherein the biologically or therapeutically active essential oil is selected from the group consisting of menthol, eucalyptol, thymol, methyl salicylate or mixtures thereof.

11. A capsule or microcapsule according to claim 10, wherein the core material, comprises:
a.) from about 0.001% to about 15% thymol;
b.) from about 0.001% to about 15% eucalyptol;
c.) from about 0.001% to about 15% methyl salicylate; and
d.) from about 0.1% to about 25% menthol.

12. A capsule or microcapsule according to claim 1, further comprising a sweetening component selected from the group consisting sucrose, glucose, dextrose, invert sugar, fructose, saccharin, cyclamic acid, aspartame, dihydrochalcone compounds, glycyrrhizin, Stevia Rebaudiana, dipotassium glycyrrhizin, chloro derivatives of sucrose; dihydroflavinol; hydroxyguaiacol esters, L-amino dicarboxylic acid gem-diamines, L-aminodicarboxylic acid aminoalkenoic acid ester amides, sorbitol, sorbitol syrup, mannitol, hydrogenated starch hydrolysate, acesulfame, L-alpha-Aspartyl-N-(2,2,4,4-tatramethyl-3-thietanyl)-D-alaninamide hydrate, chlorodeoxysucrose derivatives and mixtures thereof.

13. A capsule or microcapsule according to claim 12, wherein the chlorodeoxysucrose derivative is sucralose.

14. A capsule or microcapsule according to claim 1, further comprising a fluoride source selected from the group consisting of aminefluorides, alkali metal, alkaline earth metal, and heavy metal salts, for example, sodium fluoride, potassium fluoride, ammonium fluoride, cuprous fluoride, zinc fluoride, stannic fluoride, stannous fluoride, barium fluoride, sodium fluorozirconate, sodium monofluorophosphate, aluminum mono- and difluorophosphate, fluorinated sodium calcium pyrophosphate, acidulated monofluorophosphate and mixtures thereof.

15. A capsule or microcapsule according to claim 1, wherein the capsule or microcapsule dissolves in less than about 60 seconds.

16. A capsule or microcapsule according to claim 1, wherein the capsule or microcapsule dissolves in less than about 30 seconds.

17. A capsule or microcapsule, comprising:
a. a shell; and
b. a core, comprising:
i.) an antimicrobially effective amount of at least one antimicrobially active essential oil;
ii.) less than about 20%, by weight of the total capsule or microcapsule, of a fixed oil; and
iii.) greater than about 25%, by weight of the total capsule or microcapsule, of at least one additional essential oil.

18. A capsule or microcapsule according to claim 17, wherein the additional essential oil is present at a concentration of from about 25% to about 60%, by weight, of the composition.

19. A capsule or microcapsule according to claim 17, wherein the additional essential oil is present at a concentration of from about 15% to about 50%, by weight, of the composition.
